(19) United States
(12) Patent Application Publication
(10) Pub. No.: US 2003/0165453 A1
(43) Pub. Date: Sep. 4, 2003

(54) Methods for protecting keratinous fibers comprising applying compositions comprising at least one silicone and at least one film forming polymer

(76) Inventors: Nghi Van Nguyen, Edison, NJ (US); Hitendra Mathur, Woodbridge, NJ (US); David W. Cannell, Plainfield, NJ (US)

Correspondence Address: Finnegan, Henderson, Farabow,, Garrett & Dunner, L.L.P., 1300 I Street, N.W., Washington, DC, 20005-3315, US

(21) Appl. No.: 10/352,055

(22) Filed: Jan. 28, 2003

Related U.S. Application Data
(60) Provisional application No. 60/351,418, filed on Jan. 28, 2002.

(51) Int. Cl. A61K 7/06; A61K 7/11

(52) U.S. Cl. 424/70.12

(57) ABSTRACT

Methods and kits for protecting at least one keratinous fiber, including human keratinous fibers, comprising applying to the at least one keratinous fiber a composition comprising at least one silicone and at least one film forming polymer.

This application is based upon and claims the benefit of priority of U.S. provisional application No. 60/351,418, filed Jan. 28, 2002, the disclosure of which is incorporated herein by reference.

The present invention relates to methods for protecting at least one keratinous fiber, including human keratinous fibers, comprising applying to the at least one keratinous fiber a composition comprising at least one silicone and at least one film forming polymer.

The effect of hard water, which contains polyvalent cations, on hair is well known in the art. For example, when hair is washed in hard water, calcium ions, Ca²⁺, in the hard water may interfere with the surfactant system of the shampoo. This may result in flocculation and deposition of soil particles on the hair. See C. Zviak, The Science of Hair Care, pp. 55 (1986). This flocculation and deposition of soil particles on the hair may result in a build up on the hair surface which renders the hair at least partially resistant to subsequent chemical treatment. Thus, for example, perming such hair may be more difficult, and further for example, the hair may be more resistant to bleaching and/or coloring.

Further, alkaline earth metal salts form precipitates with certain surfactants, such as those in detergents, and these precipitates may be deposited on the hair surface. This deposition may render the hair dull and flat, and the hair may have a rough texture. Id. at pp. 57. Further, the hair may obtain a brassy golden tone.

Finally, the greening effect of hair which has been in contact with chlorinated water, such as that of swimming pools, is well known. This effect is due to the absorption of copper ions, Cu²⁺, by the hair. See G. R. Bhat, E. R. Lukenbach, R. R. Kennedy, and R. M. Parreira, The Green Hair Problem: A Preliminary Investigation, J. Soc. Cosm. Chem., pp. 1-8, Vol. 30 (1979).

Accordingly, there is a need for methods to protect hair from at least one of the aforementioned effects, such as, for example, by inhibiting the hair from contacting and/or absorbing at least one type of polyvalent cation.

Certain silicone compounds have been used as conditioning agents. Generally, these compounds lack durability on the hair shaft as they are generally hydrophobic. Accordingly, silicone compounds may not be deposited on the hair, or may be easily removed due to their weak hydrophobic interactions with the keratinous material of the hair.

The present invention, in one embodiment, provides a method for protecting at least one keratinous fiber comprising applying to the at least one keratinous fiber a composition comprising at least one silicone and at least one film forming polymer, wherein said at least one silicone and at least one film forming polymer are present in a combined amount effective to protect said at least one keratinous fiber from at least one entity. In one embodiment, the at least one entity is chosen from compositions, compounds, and atoms. In one embodiment, the at least one entity is chosen from at least one type of polyvalent cation.

In another embodiment, the invention provides a method for protecting at least one keratinous fiber comprising applying to the at least one keratinous fiber a composition comprising at least one silicone and at least one film forming polymer, wherein the at least one silicone and at least one film forming polymer are present in a combined amount effective to protect the at least one keratinous fiber from at least one type of polyvalent cation. In one embodiment, the at least one silicone and at least one film forming polymer are present in a combined amount synergistically effective to protect the at least one keratinous fiber from at least one entity, such as at least one type of polyvalent cation. In another embodiment, the at least one silicone and at least one film forming polymer are present in a combined amount effective to durably protect the at least one keratinous fiber from at least one entity, such as at least one type of polyvalent cation.

The inventors have envisaged the application to at least one keratinous fiber of a composition comprising at least one silicone and at least one film forming polymer. In particular, the inventors have discovered that such application may protect the at least one keratinous fiber, for example, by inhibiting the hair from contacting and/or absorbing at least one entity, such as at least one type of polyvalent cation.

In one embodiment, the at least one keratinous fiber may be rinsed after the application. Further, the inventive compositions may impart durable protection to the at least one keratinous fiber from at least one type of polyvalent cation.

In another embodiment, the present invention provides a kit for protecting at least one keratinous fiber comprising at least two compartments, wherein a first compartment comprises a first composition comprising at least one silicone, and wherein a second compartment comprises a second composition comprising at least one film forming polymer.

Certain terms used herein are defined below:

“At least one” as used herein means one or more and thus includes individual components as well as mixtures/combinations.

“Film forming polymer” as used herein means a polymer which, by itself or in the presence of a film-forming auxiliary, is capable, after dissolution in at least one solvent, of forming a film on the substrate to which it is applied once the at least one solvent evaporates.

“Keratinous fiber” as defined herein may be human keratinous fibers, and may be chosen from, for example, hair, eyelashes, and eyebrows.

“Polymer” as defined herein comprises copolymers formed from at least two different types of monomers and homopolymers, including but not limited to, for example, block polymers, cross linked polymers, and graft polymers.

“Polyvalent cations,” as used herein, refers to cations having more than one valence.

“Protecting” as used herein refers to inhibiting, including preventing, contact with at least one entity chosen from compositions, compounds, and atoms (including ions e.g., at least one type of polyvalent cation) and/or inhibiting, including preventing, absorption of at least one entity chosen from compositions, compounds, and atoms (including ions e.g., at least one type of polyvalent cation). Thus, for example, at least one keratinous fiber is protected when less of the at least one entity comes in contact with keratinous fiber than if the keratinous fiber was not treated. Further, for example, a keratinous fiber is protected by a composition comprising at least one silicone and at least one film forming polymer when deposition of the at least one entity is prevented and/or inhibited, wherein the protection may render the hair at least one of shiny, bright, smooth, soft, and full-bodied. Further, for example, “protecting” as used herein may include the prevention and/or inhibition of the aforementioned greening effect of hair which has been in contact with chlorinated water, such as that of swimming pools.

The level of protection from absorption of at least one entity on hair can be evaluated by measuring, and comparing, the amount of the at least one entity, for example, at least one type polyvalent cation, remaining in solution after a sample of hair has been immersed in an at least one entity-containing solution. For example, see Examples 1 and 2. Thus, whether a composition comprising at least one silicone and at least one film forming polymer is protecting at least one keratinous fiber can be determined by whether or not less of the at least one entity is absorbed by the at least one keratinous fiber treated with such a composition as compared to at least one keratinous fiber which was not treated with such a composition.

“Durable protection” as used herein, means that, following at least one shampoo after treatment, treated hair remains in a more protected state as compared to untreated hair.

“Silicone” as used herein, includes, for example, silica, silanes, siloxanes, organosilanes and organosiloxanes; and refers to a compound comprising at least one silicon atom; wherein the silicone may be chosen from linear silicones, branched silicones, and cyclic silicones; further wherein the silicone may optionally be substituted; further wherein the silicone may optionally further comprise at least one heteroatom intercalated in the silicone chain, wherein the at least one heteroatom is different from the at least one silicon atom; and further wherein the silicone may be volatile or nonvolatile.

“Substituted,” as used herein, means comprising at least one substituent. Non-limiting examples of substituents include atoms, such as oxygen atoms and nitrogen atoms, as well as functional groups, such as hydroxyl groups, ether groups, oxyalkylene groups, polyoxyalkylene groups, carboxylic acid groups, amine groups, amide groups, halogen containing groups, ester groups, siloxane groups, and polysiloxane groups.

“Synergy,” as used herein, refers to the phenomenon in which the effect of at least two components (i.e., the at least one silicone and the at least one film forming polymer) is more than additive, i.e., the effect observed with the at least two components is greater than that observed for either component alone.

It is to be understood that both the foregoing general description and the following detailed description are exemplary and explanatory only and are not restrictive of the invention as claimed. Reference will now be made in detail to exemplary embodiments of the present invention.

It has unexpectedly been discovered that a composition comprising at least one silicone and at least one film forming polymer may have properties that make it particularly desirable for use on keratinous fibers. In one embodiment, the at least one film forming polymer is not a silicone polymer. For example, with respect to hair, such as human hair, compositions comprising at least one silicone and at least one film forming polymer were found to protect the hair. Further, these compositions may impart to the at least one keratinous fiber a durable protection, i.e., durably protect the at least one keratinous fiber, even after shampooing the at least one keratinous fiber subsequent to treatment with a composition of the invention.

While not to be bound by theory, it is possible, that under certain conditions the at least one film forming polymer and the at least one silicone form a complex. A “complex,” as used herein, refers to an entity comprising at least two different types of molecules held together at least by hydrophobic forces. Thus, a complex is formed via chemical reaction of the at least two different types of molecules, wherein “chemical reaction” includes spontaneous chemical reactions and induced chemical reactions, and also includes formation of hydrophobic interactions. A complex of the at least one film forming polymer and the at least one silicone may, of course, comprise further components.

Thus, it is possible that the at least one film forming polymer and the at least one silicone form a complex, for example, through at least hydrophobic interactions between the at least one film forming polymer and the at least one silicone. The complex may be anchored to the hair surface via the portion of the complex constituted by the at least one film former polymer. The complex, once anchored to the hair, may form a barrier, inhibiting and/or preventing absorption of polyvalent cations onto the hair. The silicone portion of the inventive complex may strengthen the barrier by repulsing the hydrophilic polyvalent cations with the hydrophobicity of the at least one silicone. The overall effect may be that the inventive complex prevents and/or alleviates at least some of the absorption of polyvalent cations on the hair surface, and may thereby prevent and/or alleviate at least one of the aforementioned undesirable effects of such absorption on hair.

According to the present invention, the at least one silicone may be chosen from water-soluble silicones, oil soluble silicones, and silicones which are soluble in organic solvents. Further, the at least one silicone may be chosen from linear volatile silicones, branched volatile silicones, cyclic volatile silicones, linear nonvolatile silicones, branched nonvolatile silicones, and cyclic nonvolatile silicones.

Non-limiting examples of the at least one silicone according to the present invention include dimethicones (polydimethylsiloxanes), polyalkyl siloxanes, polyaryl siloxanes, polyalkylaryl siloxanes, polyether siloxane copolymers, alkylaryl-diaryl siloxane copolymers, dimethiconols, polydialkylsiloxane/alkylvinylsiloxane copolymers, (polydialkylsiloxane)(diphenyl)(methylvinylsiloxane) copolymers, and cyclomethicones. In one embodiment, the at least one silicone is chosen from dimethicone. Non-limiting examples of dimethicone which may be used in the composition according to the present invention are products sold under the name DC Fluid commercially available from Dow Corning. A suitable example of DC Fluids is DC 200 Fluid (1000 cst).

In one embodiment, the at least one silicone is present in the composition in an amount ranging from 0.01% to 50% by weight relative to the total weight of the composition. In another embodiment, the at least one silicone is present in an amount ranging from 0.1% to 10% by weight relative to the total weight of the composition.

According to the present invention, the at least one film forming polymer may be chosen from cationic polymers, anionic polymers, nonionic polymers, amphoteric polymers, and zwitterionic polymers. Non-limiting examples of the at least one film forming polymer are those listed at pages 1744 to 1747 of the CTFA International Cosmetic Ingredient Dictionary and Handbook, 8^{th} Edition (2000). In one embodiment, the at least one film forming polymer may be chosen from water soluble compounds, oil soluble compounds and compounds soluble in organic solvents.

According to the present invention, the at least one film forming polymer may be present in an amount generally ranging from 0.01% to 30% of active material by weight relative to the total weight of the composition, such as from 0.1% to 10% of active material by weight. One of ordinary skill in the art will recognize that the at least one film forming polymer according to the present invention may be commercially available, and may come from suppliers in the form of a dilute solution. The amounts of the at least one film forming polymer disclosed herein therefore reflect the weight percent of active material.

Non-limiting examples of the at least one film forming polymer include copolymers derived from (i) at least one vinyl monomer comprising at least one quaternary ammonium group and (ii) at least one additional monomer chosen from acrylamide, methacrylamide, alkyl acrylamides, dialkyl acrylamides, alkyl methacrylamides, dialkyl methacrylamides, alkyl acrylate, alkyl methacrylate, vinyl caprolactone, vinyl pyrrolidone, vinyl esters, vinyl alcohol, maleic anhydride, propylene glycol, and ethylene glycol.

Further non-limiting examples of the at least one film forming polymer include:

vinyl acetate/vinyl tert butylbenzoate/crotonic acid terpolymers such as those described in U.S. Pat. No. 4,282,203, the disclosure of which is incorporated herein by reference;

N-octylacrylamide/methyl methacrylate/hydroxypropyl methacrylate/acrylic acid/tert-butylamino-ethyl methacrylate copolymers such as those sold by NATIONAL STARCH under the name “AMPHOMER LV-71”;

corn starch/polyvinylpyrrolidone copolymers such as Corn Starch Modified sold by National Starch and Chemicals under the name Amaze®;

vinylpyrrolidone/vinyl acetate copolymers such as those sold by BASF under the name “LUVISKOL VA 64 Powder”;

vinyl acetate/crotonic acid/vinyl neodecanoate terpolymers such as those sold by NATIONAL STARCH under the name “RESYN® 28-2930”;

acrylic acid/ethyl acrylate/N-tert-butylacrylamide terpolymers such as those sold by BASF under the name “ULTRAHOLD 8”;

acrylic acid/acrylates/hydroxyacrylates/succinic acid copolymers such as Acrylates/C1-2 succinates hydroxyacrylates copolymer sold by ISP as ALLIANZ LT-120;

vinyl acetate/crotonic acid (90/10) copolymers such as those sold by BASF under the name “LUVISET CA 66”;

acrylic acid/methacrylic acid/acrylates/methacrylates copolymers such as Acrylates Copolymer sold by Amerchol Corp. (Edison, N.J., USA); and

vinylcaprolactam/vinylpyrrolidone/dimethylamino ethyl methacrylate copolymers such as those sold by GAF under the name “POLYMER ACP-1018”.

Further non-limiting examples of the at least one film forming polymer include:

copolymers derived from (i) 1-vinyl-2-pyrrolidone and (ii) 1-vinyl-3-methylimidazolium salt (CTFA designation: polyquaternium-16), which is commercially available from BASF Corporation under the LUVIQUAT tradename (e.g., LUVIQUAT FC 370);

copolymers derived from reaction of (i) vinylcaprolactam and (ii) vinylpyrroldone with methylvinylimidazolium methosulfate, (CTFA designation: polyquaternium-46), which is commercially available from BASF;

copolymers derived from (i) vinylpyrrolidone and (ii) quaternized imidazoline monomers (CTFA designation: polyquaternium-44), which is commercially available from BASF;

copolymers derived from (i) 1-vinyl-2-pyrrolidone and (ii) 1-vinyl-3-methylimidazolium salt (CTFA designation: polyquaternium-16), which is commercially available from BASF Corporation under the LUVIQUAT tradename (e.g., LUVIQUAT FC 370);

poly(vinylamine), optionally quaternized;

poly-4-vinyl pyridine, optionally quaternized;

poly(ethyleneimine), optionally quaternized;

dimethyldiallylammonium chloride homopolymer (CTFA designation: polyquaternium-6);

copolymers derived from (i) acrylamide and (ii) dimethyldiallylammonium chloride (CTFA designation: polyquaternium-7);

copolymers derived from (i) dimethyldiallylammonium chloride and (ii) sodium acrylate (CTFA designation: polyquaternium-22); and

terpolymers derived from (i) dimethyldiallylammonium chloride, (ii) acrylic amide and (iii) sodium acrylate (CTFA designation: polyquaternium-39).

Other non-limiting examples of the at least one film forming polymer include derivatives of polysaccharide polymers such as cationic cellulose derivatives, for example, cationic cellulose, which is available from Amerchol Corp. (Edison, N.J., USA) in their Polymer JR™, LR™ and SR™ series of polymers, as salts of hydroxyethyl cellulose reacted with trimethyl ammonium substituted epoxide (CTFA designation: polyquaternium-10); polymeric quaternary ammonium salts of hydroxyethyl cellulose reacted with lauryl dimethyl ammonium-substituted epoxide (CTFA designation: polyquaternium-24), which is available from Amerchol Corp. (Edison, N.J., USA) under the tradename Polymer LM-200™; and cationic starch and derivatives thereof, such as quaternary starch, which is available from Croda.

In one embodiment, the at least one film forming polymer is chosen from cationic polymers such as polyquaternium-16, polyquaternium-46, and polyquaternium-44. In another embodiment, the at least one film forming polymer is chosen from nonionic polymers such as copolymers derived from (1) polyvinylpyrrolidone and (2) eicosene, polymers derived from (1) corn starch and (2) polyvinylpyrrolidone; copolymers derived from (1) vinyl acetate and (2) vinylpyrrolidone. In yet another embodiment, the at least one film forming polymer is chosen from anionic polymers such as polymers derived from (1) vinyl acetate, (2) crotonic acid and (3) vinyl neodecanoate, polymers derived from (1) acrylic acid, (2) acrylates, (3) hydroxyacrylates and (4) succinic acid, and polymers derived from at least two monomers chosen from acrylic acid, methacrylic acid, esters of acrylic acid and esters of methacrylic acid. The at least one film forming polymer chosen from anionic polymers can be neutralized in order to render the anionic polymers soluble.

In one embodiment of the present invention, the at least one silicone and the at least one film forming polymer are present in the composition in a combined amount synergistically effective to protect at least one keratinous fiber by inhibiting the hair from contacting and/or absorbing at least one entity, such as at least one type of polyvalent cation.

A simple screening test exemplified in Examples 1 and 2 may be used to determine the presence of synergy and what constitutes a combined amount of the at least one silicone and the at least one film forming polymer in the composition which is synergistically effective to protect at least one keratinous fiber. The concentration of at least one type of polyvalent cation in a solution is determined prior to treatment, for example, using the Titrimetric Method for the particular multivalent ion (Hach Co., CO, USA) or ion selective electrodes. The hair may be divided into groups and treated, one group with a composition comprising both at least one silicone and at least one film forming polymer, a second group with a control solution containing the at least one silicone alone, and a third group with a control solution containing the at least one film forming polymer alone. The polyvalent cation concentration in the solution is measured after the hair has been immersed in the solution using, for example, the Titrimetric Method for the particular multivalent ion (Hach Co., CO, USA) or ion selective electrodes. A high concentration of polyvalent cation remaining in solution indicates a low polyvalent absorption to hair. The concentration of polyvalent cation remaining in solution is compared for hair treated with a composition comprising both at least one silicone and at least one film forming polymer versus untreated hair and/or hair treated with the controls to determine if synergy is observed.

The compositions of the present invention as well as those used in the methods of the present invention may be in any form, such as, for example, be in the form of a shampoo, a conditioner, a hair dye, a hair treatment, a permanent waving composition, a hair relaxer, etc.

The inventive compositions may further comprise at least one solvent. Non-limiting examples of the at least one solvent include water and organic solvents. Non-limiting examples of organic solvents include C₁-C₄ alkanols, such as ethanol and isopropanol; glycerol; glycols and glycol ethers, such as 2-butoxyethanol, propylene glycol, propylene glycol monomethyl ether, diethylene glycol monoethyl and monomethyl ether, and aromatic alcohols, such as benzyl alcohol and phenoxyethanol, and mixtures thereof. Further, the inventive compositions may be acidic or basic. The pH of the inventive compositions may be adjusted by means of at least one agent, such as acidifying agents and basifying agents, for example, those which are commonly employed in compositions for application to keratinous fibers.

In one embodiment, these compositions may further comprise at least one suitable additive chosen from additives commonly used in compositions for keratinous fibers. Non-limiting examples of the at least one suitable additive include anionic surfactants different from the at least one silicone and from the at least one film forming polymer, cationic surfactants different from the at least one silicone and from the at least one film forming polymer, nonionic surfactants different from the at least one silicone and from the at least one film forming polymer, amphoteric surfactants different from the at least one silicone and from the at least one film forming polymer, zwitterionic surfactants different from the at least one silicone and from the at least one film forming polymer, fragrances, penetrating agents, antioxidants, sequestering agents, opacifying agents, solubilizing agents, emollients, conditioning agents, colorants, screening agents (such as sunscreens and UV absorbers), preserving agents, vitamins, proteins, phospholipids, polymers such as thickening polymers different from the at least one silicone and from the at least one film forming polymer, conditioning agents, plant oils, mineral oils, synthetic oils and any other additive conventionally used in compositions for application to at least one keratinous fiber.

Needless to say, a person skilled in the art will take care to select the at least one suitable additive such that the advantageous properties of the composition in accordance with the invention are not, or are not substantially, adversely affected by the addition(s) envisaged.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific example are reported as precisely as possible. Any numerical value, however, inherently contains certain errors necessarily resulting from the standard deviation found in their respective testing measurements. The following examples are intended to illustrate the invention without limiting the scope as a result.

### EXAMPLES

### Example 1

### The Synergistic Effects of the Combination of at Least One Silicone and at Least One Film Forming Polymer

The following compositions, each thickened with 2.5% Sepigel 305 (Polyacrylamide/C₁₃₋₁₄ Isoparaffin/Laureth-7) by weight relative to the total weight of each composition, were prepared:

| | Solution | Solution | Solution | Solution |
|---|---|---|---|---|
| Component | 1 | 2 | 3 | 4 |
| Dimethicone* | — | — | 14% | 7% |
| PVP/Eicosene Copolymer** | — | 2% | — | 1% |

*DC Fluid (1,000 cst)
**Ganex V-220

Bleached hair (4 g) was treated with one of the above solutions (0.5 g solution/g hair) for 10 minutes at 45° C. A calcium solution was prepared by adding calcium chloride to water. The concentration of calcium cations in the calcium solution was determined to be 476 ppm, as determined by the Titrimetric Method for Calcium Hardness (Hach Co., CO, USA). The treated hair was then immersed in 100 g of calcium solution for 15 minutes. The hair was removed from the calcium solution and the amount of calcium remaining in the calcium solution was determined by the above titrimetric method. A high concentration of calcium remaining in solution indicates a low calcium absorption to hair. The results (average of duplicate runs) are shown in Table 1.

TABLE 1
Calcium Content Remaining in the Calcium Solution after Hair Immersion

| | Amount of Calcium Remaining in |
|---|---|
| Hair Treatment | Calcium Solution (ppm) |
| Untreated | 273 |
| Solution 1 | 266 |
| Solution 2 | 286 |
| Solution 3 | 284 |
| Solution 4 | 293 |

The data showed that hair treated with the inventive composition (Solution 4) comprising at least one film forming polymer and at least one silicone absorbed less calcium than hair treated with a film forming polymer or a silicone alone. (A t-test showed that the result for solution 4 was significantly different from the non-inventive compositions. The t-test used a 95% confidence level.) This was so even though the concentration of each individual component was twice the concentration of the component when included in the composition.

### Example 2

### The Efficiency of a Cosmetic Cream Comprising at Least One Silicone and at Least One Film Forming Polymer

The following creams were prepared:

| Component | Cream 1 (%) | Cream 2 (%) |
|---|---|---|
| Dimethicone* | 7 | — |
| PVP/Eicosene Copolymer** | 1 | — |
| Glycol | 2 | 2 |
| Emollients | 1.505 | 1.5 |
| Thickener | 2.5 | 2.5 |
| Conditioning agent | 0.5 | 0.5 |
| UV Absorber | 0.5 | 0.5 |
| Hydrolyzed Protein | 0.5 | 0.5 |
| Nonionic Surfactants | 0.12 | 0.07 |
| Amphoteric Surfactant | 0.1 | 0.1 |
| Phospholipid | 0.02 | 0.02 |
| Vitamin | 0.005 | 0.005 |
| Preservatives | 1.05 | 1.05 |
| Fragrance | 0.5 | 0.5 |
| Water | q.s. to 100 g | q.s. to 100 g |

*DC Fluid (1,000 cst)
**Ganex V-220

Efficiency of the Composition in Preventing Calcium Absorption

Following the procedure of Example 1, bleached hair was treated with one of the above creams (0.5 g/g hair) then immersed in 100 g of calcium solution containing 473 ppm calcium as determined by the Titrimetric Method for Calcium Hardness (Hach Co., CO, USA) for 15 minutes. The hair was removed from the calcium solution and the amount of calcium remaining in the calcium solution was determined by the above titrimetric method. A high concentration of calcium remaining in solution indicates a low calcium absorption to hair. The results (average of triplicate runs) are shown in Table 2.

TABLE 2
Calcium Content Remaining in the Calcium Solution after Hair Immersion

| | Amount of Calcium Remaining in |
|---|---|
| Hair Treatment | Calcium Solution (ppm) |
| Untreated | 252 |
| Cream 1 | 285 |
| Cream 2 | 259 |

The data showed that hair treated with the inventive composition (Cream 1) comprising at least one film forming polymer and at least one silicone absorbed less calcium than hair treated with a cream which did not contain the film forming polymer and the silicone (Cream 2).

Efficiency of the Composition in Preventing Iron Absorption

Following the procedure of Example 1, bleached hair was treated with the creams then immersed in 100 g of a solution containing 94 ppm of iron(III) for 15 minutes. Titrimetric Method for Iron (Hach Co., CO, USA) The hair was removed from the iron solution and the amount of iron remaining in the iron solution was determined by the above titrimetric method. The results (average of triplicate runs) are shown in Table 3.

TABLE 3
Iron Content Remaining in the Iron Solution after Hair Immersion

| | Amount of Iron Remaining in Iron |
|---|---|
| Hair Treatment | Solution (ppm) |
| Untreated | 11 |
| Cream 1 | 19 |
| Cream 2 | 14 |

The data showed that hair treated with the inventive composition (Cream 1) comprising at least one film forming polymer and at least one silicone absorbed less iron than hair treated with a cream which did not contain the film forming polymer and the silicone (Cream 2).

Efficiency of the Composition in Preventing Copper Absorption

Following the procedure of Example 1, bleached hair was treated with the creams then immersed in 100 g of a solution containing 500 ppm of copper(II) as determined by ion selective electrodes for 15 minutes. The hair was removed from the copper solution and the amount of copper remaining in the copper solution was again determined by ion selective electrodes. The results (average of triplicate runs) are shown in Table 4.

TABLE 4
Copper Content Remaining in the Copper Solution after Hair Immersion

| | Amount of Copper Remaining in |
|---|---|
| Hair Treatment | Copper Solution (ppm) |
| Untreated | 45 |
| Cream 1 | 222 |
| Cream 2 | 131 |

The data showed that hair treated with the inventive composition (Cream 1) comprising at least one film forming polymer and at least one silicone absorbed less copper than hair treated with a cream which did not contain the film forming polymer and the silicone (Cream 2).

What is claimed is:

1. A method for protecting at least one keratinous fiber comprising applying to the at least one keratinous fiber a composition comprising at least one silicone and at least one film forming polymer,
wherein said at least one silicone and at least one film forming polymer are present in a combined amount effective to protect said at least one keratinous fiber from compositions, compounds, and atoms.

2. The method according to claim 1, wherein said at least one keratinous fiber is chosen from hair.

3. The method according to claim 1, further comprising rinsing said at least one keratinous fiber following said application.

4. The method according to claim 1, wherein said atoms are chosen from ions.

5. The method according to claim 4, wherein said ions are chosen from polyvalent cations.

6. The method according to claim 1, wherein said at least one silicone is chosen from water-soluble silicones, oil-soluble silicones, and silicones which are soluble in organic solvents.

7. The method according to claim 6, wherein said at least one silicone is chosen from linear volatile silicones, branched volatile silicones, cyclic volatile silicones, linear nonvolatile silicones, branched nonvolatile silicones, and cyclic nonvolatile silicones.

8. The method according to claim 6, wherein said at least one silicone is chosen from dimethicones (polydimethylsiloxanes), polyalkyl siloxanes, polyaryl siloxanes, polyalkylaryl siloxanes, polyether siloxane copolymers, alkylaryl-diaryl siloxane copolymers, dimethiconols, polydialkylsiloxane/alkylvinylsiloxane copolymers, (polydialkylsiloxane)(diphenyl)(methylvinylsiloxane) copolymers, and cyclomethicones.

9. The method according to claim 1, wherein said at least one silicone is chosen from dimethicone.

10. The method according to claim 1, wherein said at least one silicone is present in said composition in an amount ranging from 0.01% to 50% by weight relative to the total weight of the composition.

11. The method according to claim 9, wherein said at least one silicone is present in an amount ranging from 0.1% to 10% by weight relative to the total weight of the composition.

12. The method according to claim 1, wherein said at least one silicone and at least one film forming polymer are present in a combined amount synergistically effective to protect the at least one keratinous fiber from at least one entity.

13. The method according to claim 1, wherein said at least one silicone and at least one film forming polymer are present in a combined amount effective to durably protect the at least one keratinous fiber from at least one entity.

14. The method according to claim 1, wherein said at least one silicone and the at least one film forming polymer are present in a combined amount synergistically effective to protect at least one keratinous fiber by inhibiting said at least one keratinous fiber from contacting and/or absorbing at least one entity.

15. The method according to claim 1, wherein said at least one film forming polymer is chosen from water soluble compounds, oil soluble compounds and compounds soluble in organic solvents.

16. The method according to claim 1, wherein said at least one film forming polymer is chosen from cationic polymers, anionic polymers, nonionic polymers, amphoteric polymers, and zwitterionic polymers.

17. The method according to claim 1, wherein said at least one film forming polymer is chosen from copolymers derived from (i) at least one vinyl monomer comprising at least one quaternary ammonium group and (ii) at least one additional monomer chosen from acrylamide, methacrylamide, alkyl acrylamides, dialkyl acrylamides, alkyl methacrylamides, dialkyl methacrylamides, alkyl acrylate, alkyl methacrylate, vinyl caprolactone, vinyl pyrrolidone, vinyl esters, vinyl alcohol, maleic anhydride, propylene glycol, and ethylene glycol.

18. The method according to claim 1, wherein said at least one film forming polymer is chosen from vinyl acetate/vinyl tert butylbenzoate/crotonic acid terpolymers; N-octylacrylamide/methyl methacrylate/hydroxypropyl methacrylate/acrylic acid/tert-butylamino-ethyl methacrylate copolymers; corn starch/polyvinylpyrrolidone copolymers; vinylpyrrolidone/vinyl acetate copolymers; vinyl acetate/crotonic acid/vinyl neodecanoate terpolymers; acrylic acid/ethyl acrylate/N-tert-butylacrylamide terpolymers; acrylic acid/acrylates/hydroxyacrylates/succinic acid copolymers; vinyl acetate/crotonic acid (90/10) copolymers; acrylic acid/methacrylic acid/acrylates/methacrylates copolymers; vinylcaprolactam/vinylpyrrolidone/dimethylamino ethyl methacrylate copolymers; copolymers derived from (i) 1-vinyl-2-pyrrolidone and (ii) 1-vinyl-3-methylimidazolium salt; copolymers derived from reaction of (i) vinylcaprolactam and (ii) vinylpyrroldone with methylvinylimidazolium methosulfate; copolymers derived from (i) vinylpyrrolidone and (ii) quaternized imidazoline monomers; copolymers derived from (i) 1-vinyl-2-pyrrolidone and (ii) 1-vinyl-3-methylimidazolium salt; poly(vinylamine); poly-4-vinyl pyridine; poly(ethyleneimine); dimethyldiallylammonium chloride homopolymer; copolymers derived from (i) acrylamide and (ii) dimethyldiallylammonium chloride; copolymers derived from (i) dimethyldiallylammonium chloride and (ii) sodium acrylate; terpolymers derived from (i) dimethyldiallylammonium chloride, (ii) acrylic amide and (iii) sodium acrylate; derivatives of polysaccharide polymers; polymeric quaternary ammonium salts of hydroxyethyl cellulose reacted with lauryl dimethyl ammonium-substituted epoxide; and cationic starch and derivatives thereof.

19. The method according to claim 18, wherein said at least one film forming polymer is chosen from quaternized poly(vinylamine), quaternized poly-4-vinyl pyridine, and quaternized poly(ethyleneimine).

20. The method according to claim 1, wherein said at least one film forming polymer is chosen from polyquaternium-16, polyquaternium-46, and polyquaternium-44.

21. The method according to claim 1, wherein said at least one film forming polymer is chosen from copolymers derived from (1) polyvinylpyrrolidone and (2) eicosene; polymers derived from (1) corn starch and (2) polyvinylpyrrolidone; and copolymers derived from (1) vinyl acetate and (2) vinylpyrrolidone.

22. The method according to claim 1, wherein said at least one film forming polymer is chosen from polymers derived from (1) vinyl acetate, (2) crotonic acid and (3) vinyl neodecanoate; polymers derived from (1) acrylic acid, (2) acrylates, (3) hydroxyacrylates and (4) succinic acid; and polymers derived from at least two monomers chosen from acrylic acid, methacrylic acid, esters of acrylic acid and esters of methacrylic acid.

23. The method according to claim 1, wherein said at least one film forming polymer is present in an amount ranging from 0.01% to 30% of active material by weight relative to the total weight of the composition.

24. The method according to claim 23, wherein said at least one film forming polymer is present in an amount ranging from 0.1% to 10% of active material by weight relative to the total weight of the composition.

25. The method according to claim 1, wherein said composition is in the form of a shampoo, a conditioner, a hair dye, a hair treatment, a permanent waving composition, or a hair relaxer.

26. The method according to claim 1, wherein said composition further comprises at least one solvent.

27. The method according to claim 26, wherein said at least one solvent is chosen from water and organic solvents.

28. The method according to claim 27, wherein said organic solvents are chosen from C₁-C₄ alkanols; glycerol; glycols; glycol ethers; and aromatic alcohols.

29. The method according to claim 1, wherein said composition further comprises at least one additive.

30. The method according to claim 1, wherein said at least one additive is chosen anionic surfactants different from the at least one silicone and from the at least one film forming polymer; cationic surfactants different from the at least one silicone and from the at least one film forming polymer; nonionic surfactants different from the at least one silicone and from the at least one film forming polymer; amphoteric surfactants different from the at least one silicone and from the at least one film forming polymer; zwitterionic surfactants different from the at least one silicone and from the at least one film forming polymer; fragrances; penetrating agents; antioxidants; sequestering agents; opacifying agents; solubilizing agents; emollients; conditioning agents; colorants; screening agents; preserving agents; vitamins; proteins; phospholipids; polymers different from the at least one silicone and from the at least one film forming polymer; conditioning agents; plant oils; mineral oils; and synthetic oils.

31. A method for protecting hair comprising
applying to the hair a composition comprising at least one silicone and at least one film forming polymer,
wherein said at least one silicone and at least one film forming polymer are present in a combined amount effective to protect said hair from compositions, compounds, and atoms.

32. A method for protecting at least one keratinous fiber comprising applying to the at least one keratinous fiber a composition comprising at least one silicone, wherein said at least one silicone is dimethicone, and at least one film forming polymer,
wherein said at least one silicone is and at least one film forming polymer are present in a combined amount effective to protect said at least one keratinous fiber from compositions, compounds, and atoms.

33. A method for protecting at least one keratinous fiber comprising
applying to the at least one keratinous fiber a composition comprising at least one silicone and at least one film forming polymer, wherein said at least one film forming polymer is polyvinylpyrrolidone/eicosene copolymer,
wherein said at least one silicone is and at least one film forming polymer are present in a combined amount effective to protect said at least one keratinous fiber from compositions, compounds, and atoms.

34. A method for protecting at least one keratinous fiber comprising
applying to the at least one keratinous fiber a composition comprising at least one silicone, wherein said at least one silicone is dimethicone, and at least one film forming polymer, wherein said at least one film forming polymer is polyvinylpyrrolidone/eicosene copolymer,
wherein said at least one silicone is and at least one film forming polymer are present in a combined amount effective to protect said at least one keratinous fiber from compositions, compounds, and atoms.

35. A method for protecting at least one keratinous fiber comprising
applying to the at least one keratinous fiber a composition comprising at least one silicone, at least one film forming polymer, and at least one screening agent,
wherein said at least one silicone is and at least one film forming polymer are present in a combined amount effective to protect said at least one keratinous fiber from compositions, compounds, and atoms.

36. A multicomponent kit for protecting at least one keratinous fiber comprising at least two compartments,
wherein a first compartment comprises a first composition comprising at least one silicone, and
wherein a second compartment comprises a second composition comprising at least one film forming polymer.
